(19) United States
(12) Patent Application Publication
(10) Pub. No.: US 2023/0251259 A1
(43) Pub. Date: Aug. 10, 2023

(54) METHOD FOR INACTIVATING SARS-COV-2 AND ITS USE FOR DETECTING ANTIBODIES

(71) Applicant: DIESSE DIAGNOSTICA SENESE S.P.A., Monteriggioni (SI) (IT)

(72) Inventors: Alessandra BROGI, Monteriggioni (SI) (IT); Veronica RICCI, Monteriggioni (SI) (IT); Silvana VERDIANI, Monteriggioni (SI) (IT)

(73) Assignee: DIESSE DIAGNOSTICA SENESE S.P.A., Monteriggioni (SI) (IT)

(21) Appl. No.: 17/999,487

(22) PCT Filed: May 19, 2021
(86) PCT No.: PCT/EP2021/063252

§ 371 Date: Nov. 21, 2022

(30) Foreign Application Priority Data
May 20, 2020 (EP) 20175656.6

(51) Int. Cl. G01N 33/569; G01N 33/68

(52) U.S. Cl. CPC G01N 33/56983; G01N 33/6854; G01N 2333/165

(57) ABSTRACT

The invention relates to a detection method using an inactivated SARS-CoV-2 virus for the detection of anti-SARS-CoV-2 antibodies in samples. The method includes incubating the biological sample with inactivated purified SARS-CoV-2 whole virus particles under suitable conditions to obtain a SARS-CoV-2 virus/antibody complex and detection of such complex.

### FIELD OF INVENTION

The invention relates to a detection method using an inactivated SARS-CoV-2 virus for the detection of antibodies in samples.

### STATE OF THE ART

A novel human coronavirus that is named severe acute respiratory syndrome coronavirus 2 (SARS-CoV-2, named also as COVID-19 syndrome) emerged in Wuhan, China, in late 2019. The associated disease COVID-19 turned into a global humanitarian crisis and is causing 2.319.066 confirmed cases with 157.970 confirmed deaths (7%) [1]. COVID-19 is a new syndrome, distinct from other diseases caused by coronaviruses, such as Severe Acute Respiratory Syndrome (SARS) and Middle East Respiratory Syndrome (MERS) [2].

Diagnostic assays for the detection of SARS-CoV-2, as stressed by World Health Organization (WHO), are important to fast and effectively find all suspected cases of COVID-19 in order to isolate and treat confirmed cases but, moreover, for tracing their contacts and avoid the virus to spread.

Diagnostic tests to confirm cases of COVID-19 are based on detection of unique sequences of virus RNA by nucleic acid amplification tests such as real-time reverse-transcription polymerase chain reaction (rRT-PCR) with confirmation by nucleic acid sequencing when necessary [3]. Serological screening and test can aid investigation of an ongoing outbreak and retrospective assessment of the attack rate or extent of an outbreak. In case of negative PCR and epidemiological link to COVID-19 infection, serology tests could support diagnosis. In addition, antibody testing could be used to verify the efficacy of a vaccine or to detect asymptomatic cases. In fact, antibodies reveal evidence of a previous or on-going infection [4].

The development of some serological immunoassays has been reported for the detection of SARS-CoV-2 viral proteins and antibodies in the serum or plasma [5]. The most widely used biomarkers for the detection of SARS-CoV-2 infection are IgM and IgG antibodies produced starting from the second week of viral infection. The IgM response occurs earlier than that of IgG, but it then decreases and disappears. On the other hand, IgGs can persist after infection for a long time and may have a protective role.

The strategy for the development of the serological immunoassay can be based on the use of individual viral antigens or on the combined use of different viral antigens. Different immunoassays for the determination of antibody levels in COVID-19 samples have been developed using as antigens SARS-CoV-2 proteins or recombinant forms of these.

SARS-CoV-2 structural proteins, used as immunoassay antigens, most often include spike (S), and nucleocapsid (N) proteins. The S protein, located on the surface of the virus, is important for the attachment to host cells and has been reported to be highly immunogenic. The N protein is involved in the transcription and replication of viral RNA, and interference with cell cycle processes of host cells. In many coronaviruses, the N protein has high immunogenic activity and is abundantly expressed during infection. The sensitivity of ELISA based on recombinant S protein for IgM detection is higher than that developed with N protein [6]. An IgA response may appear and grow early than IgM, leading to a stronger and more persistent response than IgM [7].

The specificity of the serological tests strongly depends on the type and quality of the used proteins, this because of cross-reactivity with other coronaviruses can lead to false positives results. In fact, for example, S protein of SARS-CoV-2 shares a 75% of amino acid identity with that of SARS, and about 50-60% with other common cold-causing coronaviruses [4].

In addition, it was shown that conformational changes occurring in the S protein of SARS-CoV-2 are fundamental for membrane fusion and viral entry [8,9]. Therefore, in case of conformational epitopes, the use of recombinant proteins may not be effective for the antibody detection.

The development of a diagnostic immunoassay based on the use of the inactivated whole viral antigen SARS-CoV-2 could be a solution and it is, thus, the object of this invention.

Moreover, this strategy allows the development of a diagnostic immunoassay that can be easily related to the immunofluorescent technique, which is a reference method for virology.

### SUMMARY OF THE INVENTION

The present invention relates to the preparation of an inactivated purified whole virus SARS-CoV-2 at large scale and its use for the production of a diagnostic immunoassay for detection of antibodies of class IgG and/or IgM and/or IgA associated to COVID-19 in biological samples, preferably human samples.

In the present invention it was surprisingly found that inactivated purified whole virus SARS-CoV-2, in particular of the strain 2019-nCoV/Italy-INMI1 (GenBank: SARS-CoV-2/INMI1-Isolate/2020/Italy: MT066156), has high selectivity and specificity for antibodies associated to COVID-19.

In particular the present invention is advantageous in that it is shown that:
i) antibodies specific to the inactivated purified whole virus SARS-CoV-2 are present in serum samples after disease recovery and in patients after vaccine but are not present in negative patients;
ii) all of 3 different types of antibodies, namely IgG, IgM, and IgA, can be detected in serum samples using the inactivated purified whole virus SARS-CoV-2 of the invention;
iii) given that IgG, IgM, and IgA are directed against different viral proteins, the use of the inactivated purified whole virus SARS-CoV-2 instead of single or selected recombinant viral proteins provides a more sensitive diagnostic immunoassay;
iv) the different reactivity of IgG in reducing and non-reducing conditions suggest that the reduction of the disulfide bonds of the viral proteins may alter their antigenicity.

The invention provides a method of detecting one or more anti-SARS-CoV-2 virus specific antibody in a biological sample, the method comprising:

a) incubating the biological sample with inactivated purified SARS-CoV-2 whole virus particles under suitable conditions to obtain a SARS-CoV-2 virus/antibody complex;
b) optionally washing to remove unbound material;
c) detecting said SARS-CoV-2 virus/antibody complex.

The method allows to detect in a sample antibodies against SARS-CoV-2 virus, including variants thereof, such as English, South African and Brazilian variants.

Preferably the detected anti-SARS-CoV-2 virus antibody is an IgG and/or IgM and/or an IgA antibody.

Preferably said inactivated purified SARS-CoV-2 whole virus particles are pre-immobilized to a solid support.

Preferably the solid support is a chip, column matrix material, a culture plate, a tube, a dish, a flask, a microtiter plate, a bead, microsphere, reactor vessel, wells, polystyrene, paramagnetic particle (PMP) or a latex magnetic particle (LMP) or a combination thereof, preferably the solid support is a microtiter plate.

Preferably the detecting step is performed by means of a labelled secondary antibody, preferably a monoclonal antibody.

Preferably the secondary antibody is labeled with a radioactive isotope, or an enzyme, preferably the enzyme is peroxidase, alkaline phosphatase, β Galactosidase or acetylcholinesterase.

Preferably the detecting step comprises measuring a signal from the label and comparing the signal to a control signal from a control biological sample known to be negative for SARS-CoV-2 virus antibodies.

Preferably said inactivated purified SARS-CoV-2 whole virus particles comprise at least one of, preferably all, the following proteins of SARS-CoV-2: virus spike glycoprotein, nucleocapsid protein, membrane protein, an envelope protein or an immunogenic fragment thereof. Preferably the biological sample is blood, serum, plasma, body fluid, saliva and other secretions from the subject or tissue or cell extracts.

Preferably the biological sample is obtained from the subject at least 3 days for IgM and IgA and at least 10 days for IgG after onset of symptoms of SARS-CoV-2 virus infection or after a risk of exposure to SARS-CoV-2 virus, or after the subject has recovered from SARS-CoV-2 virus infection. In an embodiment, the subject has received a vaccine against SARS-CoV-2 virus and the biological sample is obtained after administration of a vaccine dose. Said vaccine can be any vaccine against SARS-CoV-2 virus.

Preferably the method further comprises a quantification of amount of specific SARS-CoV-2 virus antibodies in the biological sample.

Preferably the amount of the SARS-CoV-2 virus antibody in the biological sample is proportional to the amount of the complex detected.

For “SARS-CoV-2” it is herein intended the severe acute respiratory syndrome coronavirus 2. This term includes all SARS-CoV-2 strains and variants. Sequences of SARS-CoV-2 virus and of its antigenic proteins are known and available in the art. Also sequences of SARS-CoV-2 variants, such as English, South African and Brazilian variants, are known in the field. The skilled person in the field knows what is intended for SARS-CoV-2 variants, in particular for English, South African and Brazilian variants.

For “anti-SARS-CoV-2 virus specific antibody” is intended an antibody which binds and recognizes in a specific manner a SARS-CoV-2 virus.

The inactivated purified SARS-CoV-2 whole virus particles can be obtained in any known manner, for example they can be obtained from an isolated biological sample from an infected subject through inactivation procedures.

It is herein disclosed a method to obtain from an isolated biological sample inactivated SARS-CoV-2 whole virus particles to use in the detection method of the invention comprising the steps of:

a) inactivating SARS-CoV-2 whole virus particles by at least two additions of an inactivating reagent to said sample and optionally
b) purifying inactivated SARS-CoV-2 whole virus particles.

Preferably, such inactivating reagent is β-propiolactone, preferably at 0.1% (v/v). Preferably the additions of 0.1% (v/v) of β-propiolactone are performed at temperature ranging from 15° C. to 37° C. for at least 1 hour, preferably for 3 hours.

The inactivated purified SARS-CoV-2 whole virus particles can be of any SARS-CoV-2 strain or variant. Preferably the SARS-CoV-2 whole virus particles belong to the strain 2019-nCoV/Italy-INMI1 (GenBank: SARS-CoV-2/INMI1-Isolate/2020/Italy: MT066156), or to a natural or recombinant derivative thereof.

The invention further provides a kit for working the method as defined above comprising:

a) a solid support coated with inactivated purified SARS-CoV-2 whole virus particles,
b) at least one labeled secondary antibody able to bind to human IgG or IgM or IgA or a labelled antibody anti-SARS-CoV-2 antigen,
c) a negative control;
d) a positive control;
e) a cut-off control or calibrator;
f) a sample diluent;
g) a substrate solution;
h) a washing solution;
i) optionally a stop solution.

The invention also provides the use of inactivated purified SARS-CoV-2 whole virus particles, preferably obtained by the inactivation method as defined above, for detecting SARS-CoV-2 antibodies wherein said SARS-CoV-2 virus is strain 2019-nCoV/Italy-INMI1 (GenBank: SARS-CoV-2/INMI1-Isolate/2020/Italy: MT066156) or natural or recombinant derivative thereof.

The method for the production of the inactivated purified whole virus SARS-CoV-2 may comprise: virus propagation in cell cultures, inactivation, and purification.

In particular, SARS-CoV-2 whole virus may be propagated in cells such as in epithelial cells (e.g. VERO cell lines and subclones), followed by inactivation and purification, in particular by sedimentation, of the inactivated SARS-CoV-2 whole virus particles to recover the whole virus particles containing the proteins.

In the present invention the biological sample may be obtained from the subject at least 3, 4, 5 or 6 days for IgM and IgA and at least 10, 11, 12, 13, 14, 15, 16, 17 or 18 days for IgG after onset of symptoms of SARS-CoV-2 virus infection.

The immunoenzymatic method of the invention may comprise the following steps:
1) coating of the inactivated purified whole virus SARS-CoV-2 to a solid support (e.g. chips, microspheres, polystyrene, reactor vessels or wells, microtiter plate);
2) addition of the human sample to be tested to the coated solid support and incubation under conditions suitable for the formation of the antigen/antibody immune binding complex;
3) optionally washing to remove any unbound material;
4) addition of the conjugated secondary antibodies wherein said conjugated secondary antibodies are against human antibodies or against a SARS-CoV-2 antigen; incubation under conditions suitable for the formation of antigen/antibody immune complexes;
5) washing to remove any unbound material;
6) detection of the presence of antigen/antibody immune binding complexes.

The invention will be illustrated by means of non-limiting examples in reference to the following figures.

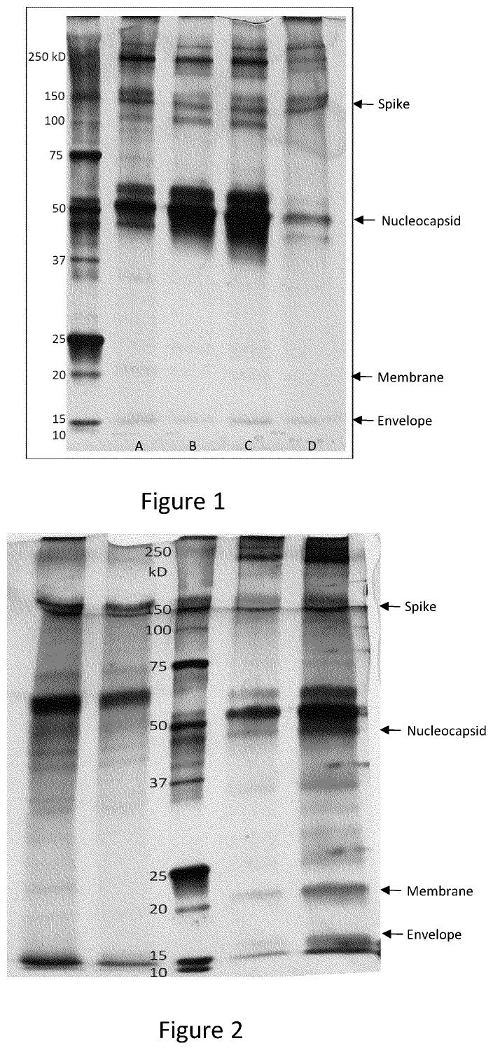

FIG. 1. Gel electrophoresis performed in non-reducing conditions with a 10% separation. Detection by silver staining. On the right of the molecular-weight size marker, are reported four different production batches of the inactivated purified whole virus SARS-CoV-2 (i.e. lanes A, B, C, and D). The predominant band is albumin resulting from cell culture medium.

FIG. 2. Gel electrophoresis performed in reducing (left of size marker), and non-reducing (right of size marker) conditions. Detection by silver staining. The gel was performed at different concentrations of the purified inactivated viral antigen SARS-CoV-2 (2 and 5 μl).

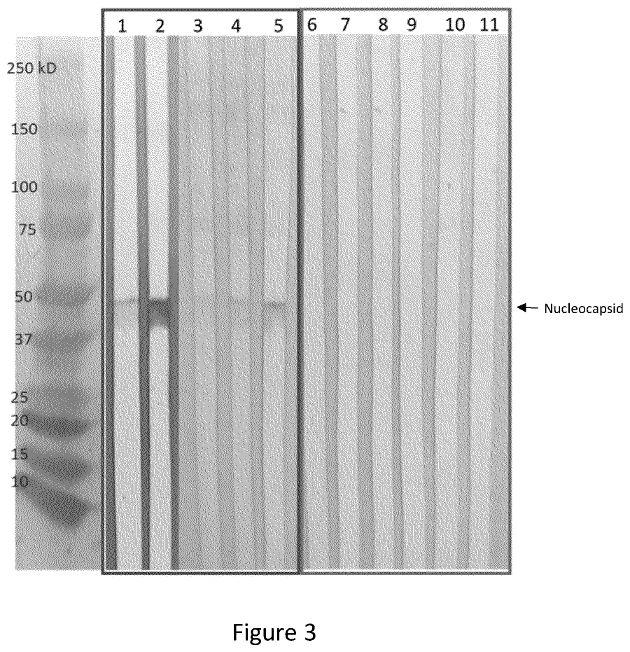

FIG. 3. Western Blot performed in reducing conditions and detected with five COVID-19 positive serum samples (lanes 1-5) and with six COVID-19 negative positive serum samples (lanes 6-11). Detection is performed with an anti-human IgG.

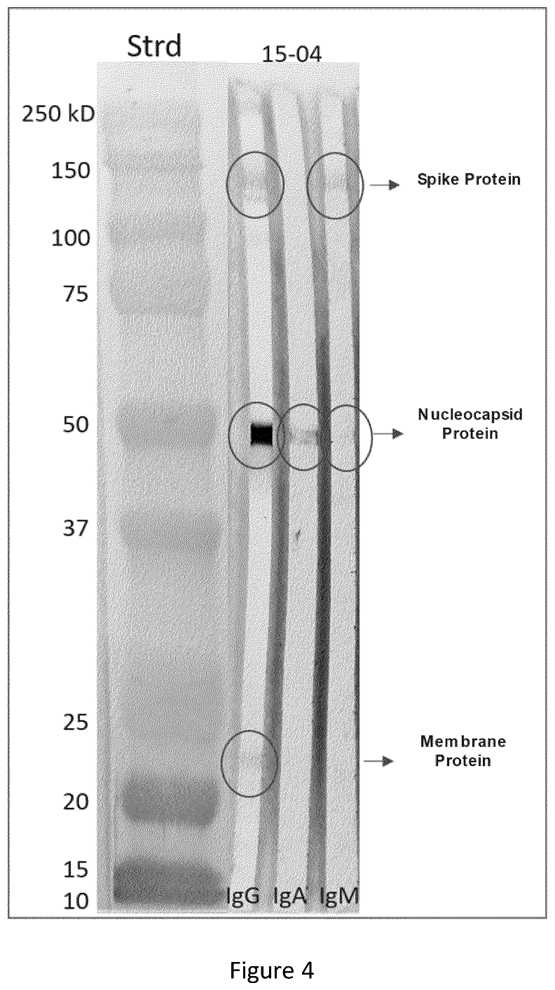

FIG. 4. Western Blot performed in non-reducing conditions and detected with one COVID-19 positive serum sample from a recovered patient. Detection is performed with anti-human IgG, IgA, and IgM.

### DETAILED DESCRIPTION OF THE INVENTION

Methods

Virus Propagation

VERO E6 cells (ATCC, CRL-1586) (cultivated and collected) are propagated by cultured with conventional techniques [10]. The cell substrates are infected with SARS-CoV-2 virus (strain 2019-nCoV/Italy-INMI1, the complete sequence was submitted to GenBank (SARS-CoV-2/INMI1-Isolate/2020/Italy: MT066156) and is available on GISAID website (BetaCoV/Italy/INMI1-isI/2020: EPI_ISL_410545) at specific multiplicity of infection MOI value of 0.05 and are incubated at 37° C. for 46-50 hours in roller bottles (850 cm² each). After incubation, the cytopathic effect (cpe) distinctive for SARS-CoV-2 is evaluated observing by microscopy on the VERO E6 cell monolayer the cell rounding, detachment, and degeneration [11]. The virus titer is quantified by using the Reed-Muench method and calculating the TCID50/ml [12]. After 48 hours the cpe is completed and viruses are harvested and frozen at −80° C. Samples are taken to evaluate the infectious titer of the virus.

Virus Inactivation

After thawing, the viruses are inactivated with β-propiolactone (Natalex, CAS 57-57-8). To ensure inactivation, at least two consecutive additions of 0.1% [v/v] of β-propiolactone are added to the fixed volume of the viral suspension. Each cycle of β-propiolactone treatment is incubated at room temperature for 3 hours. Then, the viral suspension is incubated for 2 hours at 37° C. The inactivation control is checked by verifying the disappearance of live viruses through cpe on a sub-culture of inactivated material. This control is performed using live viruses with a 10⁴-10⁵ TCID50/ml as reference. By microscopy the presence/absence of cpe is observed and the TCID50/ml titer is calculated. The virus is inactivated, if the VERO E6 cell monolayer does not show the SARS-CoV-2 cpe and the TCID50/ml is <1*10^{1,5}. In case of incomplete inactivation, the treatment with 0.1% of β-propiolactone is repeated and checked. The inactivated viruses are frozen at −80° C.

Purification

Purification of the inactivated viruses is performed first by clarification by centrifugation at 10.000 g to remove cell debris. The supernatant is collected and concentrated by ultracentrifugation at 100.000 g. The inactivated viruses are present in the pellet, the supernatant is collected and the pellet is sonicated and resuspended in PBS (pH 7.2-7.8). The inactivated purified viruses are distributed in vials and frozen at −80° C.

Immunoenzymatic Method

The immunoenzymatic method of the present invention for the diagnosis of COVID-19 in a subject comprises the step of detecting antibodies specific for SARS-CoV-2 in a human sample by reacting under proper conditions said human sample with inactivated purified whole virus SARS-CoV-2 to produce an immunocomplex, which is then detected by a conjugated secondary antibody.

In an embodiment, the immunoenzymatic method of the invention comprises the following steps:
1) coating of the inactivated purified whole virus SARS-CoV-2 to a solid support (e.g. chips, microspheres, polystyrene, reactor vessels or wells, microtiter plate);
2) addition of the human sample to be tested to the coated solid support and incubation under conditions suitable for the formation of the antigen/antibody immune binding complex;
3) optional washing to remove any unbound material;
4) addition of the conjugated secondary antibodies wherein said conjugated secondary antibodies are against human antibodies or against a SARS-CoV-2 antigen and incubation under conditions suitable for the formation of antigen/antibody immune complexes;
5) washing to remove any unbound material;
6) detection of the presence of antigen/antibody immune binding complexes wherein the antibody is specific for SARS-CoV-2.

Suitable conditions for the formation of the antigen/antibody immune binding complex (step 2) are for example incubation at a temperature comprised between 18 and 37° C., e.g. room temperature, for a period comprised between 0.25 and 1 hour.

In an embodiment, the inactivated purified whole virus SARS-CoV-2 (1:100) is coated to the microtiter plates in citrate buffer (pH 2.9-3.1) by incubating at room temperature for 24 hours. After incubation, the plates are washed, treated with a saturating agent (e.g. buffered solution of PBS at 7.2-7.4 pH containing 6-7% sucrose and 0.5-1.0% BSA), and then dried. The human sample diluted in a range between 1:4-1:100 is applied to the microtiter plate and is incubated at room temperature, such as 18-37° C., for 0.25-1 hour. After incubation and the optional washings, the conjugated secondary antibody is added to the microtiter plate and incubated at room temperature, such as 18-37° C., for 0.25-1 hour. After incubation and washings, detection is performed depending on the used immunoenzymatic technique (e.g. addition of substrate and stop solution to read at 405-450 nm or 620 nm in case of ELISA). The results of the test are calculated by dividing the absorbance of each sample by the absorbance of the cut-off value (i.e. OD mean of negative sera plus two standard deviations). Qualitative results are expressed as index (OD sample/OD cutoff). Results are positive if the index is above 1.1. Quantitative results are expressed as Binding Antibody Units (BAU/ml).

Preferably, said inactivated purified SARS-CoV-2 whole virus particles are particles of the SARS-CoV-2 strain 2019-nCoV/Italy-INMI1 (GenBank: SARS-CoV-2/INMI1-Isolate/2020/Italy: MT066156) or natural or recombinant derivative thereof.

The secondary antibody may be an:
anti-human-IgG or/and -IgM or/and -IgA antibody;
or
an anti-SARS-CoV-2 antigen antibody.

Said anti-human-IgG or/and -IgM or/and -IgA antibody can be any commercially available secondary anti-human-IgG or/and -IgM or/and -IgA antibody.

Said anti-SARS-CoV-2 antigen antibody can be an antibody directed to any antigen of SARS-CoV-2, for example directed to the Spike protein, e.g. S1. Typically, it is a human antibody. Typically, it is an antibody directed to the inactivated SARS-CoV-2, or an antigenic portion thereof, coated on said solid support.

In an embodiment, said antibody anti-SARS-CoV-2 antigen has a neutralizing activity, in particular a neutralizing activity against the SARS-CoV-2 and its variants, such as for example the English, the South African and the Brazilian variant; in this embodiment, the method advantageously allows to identify and quantify antibodies present in the human sample having a neutralizing activity against the SARS-CoV-2 virus or variants thereof. Such antibody anti-SARS-CoV-2 antigen can be a commercially available antibody directed to an antigen of SARS-CoV-2, for example the A514 antibody by Proteogenix directed to the SARS-CoV-2 Spike protein 51. Different secondary antibodies can be used to detect the presence of specific antibodies in the sample and the relative amounts of different antibodies.

The diagnostic kits for the detection of antibodies specific for SARS-CoV-2 may be based on an immunoenzymatic assay comprising the following reagents:
a solid support, such as a microtiter plate, coated with the inactivated purified whole virus SARS-CoV-2;
a labelled anti-human antibody, for instance an enzyme-conjugated anti-human antibody (e.g. such as any monoclonal or polyclonal antibodies anti-human or other species IgG, IgM, and/or IgA labeled with horseradish peroxidase in 2% newborn calf serum in PBS solution), or a labelled antibody anti-SARS-CoV-2 antigen, for instance an enzyme-conjugated antibody (e.g. such as any monoclonal or polyclonal antibodies anti-SARS-CoV-2 antigen labeled with horseradish peroxidase in 2% newborn calf serum in PBS solution);
a negative control (for instance 1% BSA in PBS solution containing 0.2% Tween-20);
a positive control (for instance specific anti-SARS-CoV-2 monoclonal/polyclonal antibodies in PBS solution in containing 1% BSA and 0.2% Tween-20);
a cut-off control or calibrator (for instance specific anti-SARS-CoV-2 monoclonal/polyclonal antibodies in PBS solution in containing 1% BSA and 0.2% Tween-20);
a sample diluent (for instance 2% newborn calf serum in PBS solution containing 0.5%

Brij);
a washing buffer (for instance a PBS solution containing 0.5% Brij);
a substrate (for instance a solution of 3,3′,5,5′-tetramethylbenzidine in 0.05M citrate buffer);
optionally a stop solution (for instance 0.3 M H₂SO₄).

The kit can be for manual or automatable applications. For example it can be in the form of a disposable device for use for the automatic performance of immunoenzymatic assays in suitable instruments, such as for example the Chorus and Chorus Trio devices from DIESSE Diagnostica Senese S.p.A.

Electrophoresis

One-dimensional SDS-PAGE was performed with precasted gel (Bio-Rad) with a 10% separation. All samples were prepared by dilution with an equal volume of Laemmli sample buffer (Bio-Rad, 1610737). In case of reducing conditions 2-mercaptoethanol is added to the Laemmli buffer and the sample is heated at 95° C. for 5 minutes. Precision Plus Protein™ unstained standards (Bio-Rad) were used as molecular-weight markers. Gels were run in electrophoresis cell at 200 V until the dye front reached the bottom of the gel. The running buffer used was Tris/glycine/SDS (25 mM Tris, 192 mM glycine, 0.1% [w/v] SDS). Gels were stained with silver staining (Pierce Silver Stain Kit).

Western Blot

The proteins separated on SDS-PAGE were transferred onto nitrocellulose (Whatman) by wet blotting for 1 hour at 100 V. The nitrocellulose membranes were blocked (5% skimmed milk in tris-buffered saline) for 3 hours, incubated overnight at 4° C. with serum samples diluted in blocking buffer with 0.05% Tween-20, washed 3 times with tris-buffered saline with 0.05% Tween-20, incubated for 1 hour with either anti-IgG, anti-IgM or anti-IgA antibody conjugated with alkaline phosphatase in blocking buffer with 0.05% Tween-20, washed 3 times with tris-buffered saline with 0.05% Tween-20, incubated until detection with BCIP/NBT substrate solution, and stopped with water.

Sensitivity and Specificity

The sensitivity is calculated as the % ratio between the number of true positive samples and the sum of the number of true positives plus the number of false negatives. The specificity is calculated as the % ratio between the number of true negative samples and the sum of the number of true negatives plus the number of false positives.

$$sensitivity = \frac{number of true positives}{number of true positives + number of false negatives}$$

$$specificity = \frac{number of true negatives}{number of true negatives + number of false positives}$$

In a work from Padoan et al. (2020) [13] the analytical and clinical performances of an assay according to the method of the present invention (Diesse ENZY-WELL SARS-CoV-2 IgG) were evaluated and compared to four commercially available chemiluminescent assays (Abbott SARS-Cov-2 IgG, Roche Elecsys anti-SARS-CoV-2, Ortho SARS-CoV-2 total and IgG). Overall, there was a good agreement between all assays, one of the better result being obtained for the assay according to the invention. Furthermore, the strongest PRNT₅₀ correlation with antibody levels was obtained with the assay of the invention, showing the efficacy of this assay to detect neutralizing antibodies.

Also the work of Nuccetelli et al. (2020) [14] evaluates an assay according to the method of the present invention, in particular a simultaneous anti-SARS-CoV-2 IgA/IgG/IgM immunoassay, performed on an automated instrument by ELISA kit coated with inactivated SARS-CoV-2 (“Chorus SARS-CoV-2 Screen Serum”). Specificities and sensitivities results were excellent, giving values of 100% for both parameters, leading to a small number of false positive or false negative individuals, that is crucial for a broad population screening. The authors found also that the choice to use whole virus particles proved to be much more effective than recombinant specific antigens, indeed the test is able to identify more positive samples than kits using recombinant antigens. The use of such virus epitopes as well as simultaneous anti-SARSCoV-2 IgA/IgM/IgG detection was encouraged to help to contain COVID-19 spreading.

### EXAMPLES

### Example 1

Reactivity of the Inactivated Purified Whole Virus SARS-CoV-2

The production process of the inactivated purified whole virus SARS-CoV-2 was optimized by setting up the conditions (e.g. β-propiolactone concentration between 0.05 and 0.5% [v/v], inactivation incubation times between 1 and 24 h, inactivation temperatures between 15° C. and 37° C., centrifugation with/without sonication). The optimized process was verified by checking:
the presence of the relevant proteins (i.e. spike glycoprotein, nucleocapsid protein, membrane protein, and envelope protein),
the reactivity of the viral proteins in reducing and non-reducing conditions to reveal the role of conformation in antibody detection,
the reactivity of the viral proteins in detecting antibodies in COVID-19 positive and negative samples.

First, the efficacy and the reproducibility of the production process of the inactivated purified whole virus SARS-CoV-2 were evaluated by electrophoresis (FIG. 1). Four different batches of production (i.e. lanes A, B, C, and D) were compared demonstrating the reproducibility of the method, which is reliable both in terms of total protein concentration and of protein reactivity. The electrophoresis demonstrates the presence of the four proteins: spike glycoprotein (i.e. amino acid mass≈140 kDa), nucleocapsid protein (i.e. amino acid mass≈46 kDa), membrane protein (i.e. amino acid mass≈24 kDa), and envelope protein (i.e. amino acid mass≈8 kDa). The molecular weight difference of the viral proteins between reducing and non-reducing conditions was evaluated by electrophoresis (FIG. 2). The electrophoresis in non-reducing conditions allows the detection of the four viral proteins, on the contrary in reducing conditions not all the four viral proteins can be observed.

The reactivity of the viral proteins in detecting antibodies in COVID-19 positive and negative serum samples was evaluated by analyzing the inactivated purified whole virus SARS-CoV-2 in Western Blot (FIG. 3). Results demonstrated that the negative serum samples do not contain any antibody specific for SARS-CoV-2. The positive samples demonstrate that, in the reducing conditions of the electrophoresis, only the nucleocapsid protein is recognized.

The reactivity of the viral proteins present in the inactivated purified whole virus SARS-CoV-2 was further analyzed by Western Blot in non-reducing conditions (FIG. 4). Results demonstrate that in non-reducing conditions, in addition to the nucleocapsid protein, also the spike glycoprotein and the membrane protein are recognized by the IgG present in a COVID-19 positive serum sample from a patient declared recovered after 2 negative RT-PCR. IgM antibodies detect the nucleocapsid protein and the spike glycoprotein. IgA antibodies detect only the nucleocapsid protein.

These results demonstrate that:
antibodies specific to the inactivated purified whole virus SARS-CoV-2 are present in serum samples after disease recovery and in patients but are not present in negatives;
the 3 different types of antibodies IgG, IgM, and IgA can be detected in serum samples using the inactivated purified whole virus SARS-CoV-2;
IgG, IgM, and IgA are directed against different viral proteins, thus supporting the hypothesis that the use of the inactivated purified whole virus SARS-CoV-2 instead of a single recombinant viral protein can provide a more sensitive diagnostic immunoassay;
the different reactivity of IgG in reducing and non-reducing conditions may suggest that the reduction of the disulfide bonds of the viral proteins may alter their antigenicity.

### Example 2

Diagnostic Performance of the ELISA Test Based on the Inactivated Purified Whole Virus SARS-CoV-2 for the Detection of IgG Antibodies

According to the immunoenzymatic method of the invention, an ELISA test for the detection of IgG antibodies was developed. The detection was performed with an anti-human IgG monoclonal antibody labeled with horseradish peroxidase and reading the absorbance at 450 nm or 450/620 nm, using a specific substrate (3,3′,5,5′-tetramethylbenzidine, Sigma, 860336) and H₂SO₄(Sigma, 30743-M) 0.3 M as stop solution.

The diagnostic performances of the ELISA were evaluated in a clinical study comprising 495 serum samples characterized by an immunofluorescent test. Results of this comparison study are reported in Table 1 and demonstrate a strong correlation with immunofluorescence in terms of true positive (i.e. 62/67) and true negative samples (i.e. 410/428).

TABLE 1
Comparison study performed on a total of 495 serum samples
tested with the ELISA of the present invention and an immunofluorescent
test for the detection of anti-SARS-CoV-2 IgG.

| | | Reference (immunofluorescent test) | | |
|---|---|---|---|---|
| | | Positive | Negative | Total |
| ELISA | Positive | 62 | 18 | 80 |
| object of the | Negative | 5 | 410 | 415 |
| invention | Total | 67 | 428 | 495 |

The diagnostic sensitivity and specificity of the anti-SARS-CoV-2 IgG ELISA of the present invention are reported in Table 2.

TABLE 2
Diagnostic sensitivity and specificity
of the anti-SARS-CoV-2 IgG ELISA

| | | |
|---|---|---|
| Diagnostic Sensitivity | 92.5% | CI95%: 83.6-96.8 |
| Diagnostic Specificity | 95.8% | CI95%: 93.4-97.3 |

### Example 3

Diagnostic Performance of the ELISA Test Based on the Inactivated Purified Whole Virus SARS-CoV-2 for the Detection of IgM Antibodies

According to the immunoenzymatic method of the invention, an ELISA test for the detection of IgM antibodies was developed. The detection was performed with an anti-human IgM monoclonal antibodies labeled with horseradish peroxidase and reading the absorbance at 450 nm or 450/620 nm, using a specific substrate (3,3′,5,5′-tetramethylbenzidine) and H₂SO₄ 0.3 M as stop solution.

The diagnostic performances of the ELISA were evaluated in a clinical study comprising 397 serum samples characterized by an immunofluorescent test. Results of this comparison study are reported in Table 3 and demonstrate a strong correlation with immunofluorescence in terms of true positive (i.e. 57/65) and true negative samples (i.e. 322/332).

TABLE 3
Comparison study performed on a total of 397 serum samples
tested with the ELISA of the present invention and an immunofluorescent
test for the detection of anti-SARS-CoV-2 IgM.

| | | Reference (immunofluorescent test) | | |
|---|---|---|---|---|
| | | Positive | Negative | Total |
| ELISA | Positive | 57 | 10 | 67 |
| object of the | Negative | 8 | 322 | 330 |
| invention | Total | 65 | 332 | 397 |

The diagnostic sensitivity and specificity of the anti-SARS-CoV-2 IgM ELISA of the present invention are reported in Table 4.

TABLE 4
Diagnostic sensitivity and specificity
of the anti-SARS-CoV-2 IgM ELISA

| | | |
|---|---|---|
| Diagnostic Sensitivity | 87.7% | CI95%: 77.5-93.6 |
| Diagnostic Specificity | 97.0% | CI95%: 94.5-98.3 |

### Example 4

Diagnostic Performance of the ELISA Test Based on the Inactivated Purified Whole Virus SARS-CoV-2 for the Detection of IgA Antibodies

According to the immunoenzymatic method of the invention, an ELISA test for the detection of IgA antibodies was developed. The detection was performed with an anti-human IgA monoclonal antibodies labeled with peroxidase and reading the absorbance at 450 nm or 450/620 nm, using a specific substrate (3,3′,5,5′-tetramethylbenzidine) and H₂SO₄ 0.3 M as stop solution.

The diagnostic performances of the ELISA were evaluated in a clinical study comprising 463 serum samples characterized by an immunofluorescent test. Results of this comparison study are reported in Table 5 and demonstrate a strong correlation with immunofluorescence in terms of true positive (i.e. 59/63) and true negative samples (i.e. 385/400).

TABLE 5
Comparison study performed on a total of 463 serum samples
tested with the ELISA of the present invention and an immunofluorescent
test for the detection of anti-SARS-CoV-2 IgA.

| | | Reference (immunofluorescent test) | | |
|---|---|---|---|---|
| | | Positive | Negative | Total |
| ELISA | Positive | 59 | 15 | 74 |
| object of the | Negative | 4 | 385 | 389 |
| invention | Total | 63 | 400 | 463 |

The diagnostic sensitivity and specificity of the anti-SARS-CoV-2 IgA ELISA of the present invention are reported in Table 6.

TABLE 6
Diagnostic sensitivity and specificity
of the anti-SARS-CoV-2 IgA ELISA

| | | |
|---|---|---|
| Diagnostic Sensitivity | 93.7% | CI95%: 84.7-97.5 |
| Diagnostic Specificity | 96.3% | CI95%: 93.9-97.7 |

### Example 5

Diagnostic Performance of the ELISA Test Based on the Inactivated Purified Whole Virus SARS-CoV-2 for the Detection of Total Ig Antibodies

According to the immunoenzymatic method of the invention, an ELISA test for the detection of the total Ig was developed. The detection was performed with a monoclonal antibody anti-SARS-CoV-2 antigen labeled with peroxidase and reading the absorbance at 620 nm, using a specific substrate (3,3′,5,5′-tetramethylbenzidine).

The diagnostic performances of the ELISA were evaluated in a clinical study comprising 378 serum samples characterized by PCR. Results of this comparison study are reported in Table 7 and demonstrate a strong correlation with PCR in terms of true positive (i.e. 85/86) and true negative samples (i.e. 292/292).

TABLE 7
Comparison study performed on a total of 378 serum samples
tested with the ELISA of the present invention and
PCR test for the detection of anti-SARS-CoV-2 Ig.

| | | Reference (PCR test) | | |
|---|---|---|---|---|
| | | Positive | Negative | Total |
| ELISA | Positive | 85 | 0 | 85 |
| object of the | Negative | 1 | 292 | 293 |
| invention | Total | 86 | 292 | 378 |

The diagnostic sensitivity and specificity of the anti-Ig ELISA of the present invention are reported in Table 8.

TABLE 8
Diagnostic sensitivity and specificity
of the anti-SARS-CoV-2 Ig ELISA

| | | |
|---|---|---|
| Diagnostic Sensitivity | 98.8% | CI95%: 93.7-99.8 |
| Diagnostic Specificity | 100% | CI95%: 98.7-100.0 |

### Example 6

Diagnostic Performance of the ELISA Test Based on the Inactivated Purified Whole Virus SARS-CoV-2 for the Detection of Antigen-Neutralizing Ig Antibodies

According to the immunoenzymatic method of the invention, an ELISA test for the detection of antigen-neutralizing Ig was developed. The detection was performed with a monoclonal antibody anti-SARS-CoV-2 antigen labeled with peroxidase and reading the absorbance at 450 nm or 450/620 nm, using a specific substrate (3,3′,5,5′-tetramethylbenzidine) and H₂SO₄ 0.3 M as stop solution.

The diagnostic performances of the ELISA were evaluated in a clinical study comprising 1.106 serum samples characterized by PRNT (Plaque reduction neutralization test) seroneutralization. The plaque reduction neutralization test allows to quantify the titer of neutralizing antibody for a virus. These 1.106 serum samples comprise: 651 pre-pandemic samples and 455 pandemic samples. Results of this comparison study are reported in Table 9 and demonstrate a strong correlation with PRNT seroneutralization in terms of true positive (i.e. 242/243) and true negative samples (i.e. 861/863).

TABLE 9
Comparison study performed on a total of 1.106 serum
samples tested with the ELISA of the present invention
and PRNT seroneutralization test for the detection
of anti-SARS-CoV-2 antigen neutralizing Ig.

| | | Reference (PRNT seroneutralization test) | | |
|---|---|---|---|---|
| | | Positive | Negative | Total |
| ELISA | Positive | 242 | 2 | 244 |
| object of the | Negative | 1 | 861 | 862 |
| invention | Total | 243 | 863 | 1106 |

The diagnostic sensitivity and specificity of the antigen-neutralizing Ig ELISA of the present invention are reported in Table 10.

TABLE 10
Diagnostic sensitivity and specificity
of the antigen-neutralizing Ig ELISA.

| | | |
|---|---|---|
| Diagnostic Sensitivity | 99.6% | CI95%: 97.7-99.9 |
| Diagnostic Specificity | 99.8% | CI95%: 99.2-99.9 |

### Example 7

Diagnostic Performance of the ELISA Test Based on the Inactivated Purified Whole Virus SARS-CoV-2 for the Detection of Antigen-Neutralizing Ig Antibodies in Case of English Variants

According to the immunoenzymatic method of the invention, an ELISA test for the detection of antigen-neutralizing Ig was developed. The detection was performed with a monoclonal antibody anti-SARS-CoV-2 antigen labeled with peroxidase and reading the absorbance at 450 nm or 450/620 nm, using a specific substrate (3,3′,5,5′-tetramethylbenzidine) and H₂SO₄ 0.3 M as stop solution.

The diagnostic performances of the ELISA were evaluated in a clinical study comprising 56 serum samples characterized for the presence of antibodies anti-SARS-CoV-2 and its English variant by a neutralizing test. Results of this comparison study are reported in Table 11 and demonstrate a strong correlation with seroneutralization tests performed with SARS-CoV-2 INMI strain or with SARS-CoV-2 English variant. The results obtained with the ELISA object of the invention confirm the capacity of this method to detect also the English variant.

TABLE 11
Comparison study performed on a total of 56 serum samples tested
with the ELISA of the present invention and neutralization test
for the detection of anti-SARS-CoV-2 English variant Ig.

| Serum | Seroneutralization test with SARS-CoV-2 | | ELISA object of |
|---|---|---|---|
| Sample # | English variant | INMI strain | the invention |
| 1 | P | P | P |
| 2 | N | N | N |
| 3 | N | P | P |
| 4 | P | P | P |
| 5 | P | P | P |
| 6 | P | P | P |
| 7 | P | P | P |
| 8 | N | N | N |
| 9 | P | P | P |
| 10 | P | P | P |
| 11 | P | P | P |
| 12 | P | P | P |
| 13 | P | N | P |
| 14 | P | P | P |
| 15 | P | P | P |
| 16 | P | N | P |
| 17 | P | P | P |
| 18 | P | P | P |
| 19 | N | P | P |
| 20 | P | P | P |
| 21 | P | P | P |
| 22 | P | P | P |
| 23 | P | P | P |
| 24 | N | N | N |
| 25 | P | P | P |
| 26 | N | N | N |
| 27 | N | P | P |
| 28 | N | N | N |
| 29 | N | N | N |
| 30 | P | P | P |
| 31 | N | N | N |
| 32 | P | P | P |
| 33 | P | P | P |
| 34 | P | P | P |
| 35 | P | P | P |
| 36 | P | P | P |
| 37 | P | P | P |
| 38 | N | N | N |
| 39 | P | P | P |
| 40 | P | P | P |
| 41 | P | P | P |
| 42 | P | P | P |
| 43 | N | P | P |
| 44 | P | P | P |
| 45 | P | P | P |
| 46 | P | P | P |
| 47 | N | N | N |
| 48 | P | P | P |
| 49 | P | P | P |
| 50 | N | P | P |
| 51 | P | P | P |
| 52 | P | P | P |
| 53 | P | P | P |
| 54 | P | P | P |
| 55 | P | P | P |
| 56 | N | P | P |

In conclusion, we herein described a method for the production of an inactivated purified whole virus SARS-CoV-2 at large scale, which can be used for the preparation of the immunoenzymatic assays of the invention for the detection of antibodies of class IgG, IgM, and IgA in serum samples.

The detected antibodies IgG, IgM, and IgA recognize different viral proteins, thus demonstrating the importance of a diagnostic immunoassay containing all the immunogenic proteins of the whole virus SARS-CoV-2. In fact, the use of the inactivated purified whole virus SARS-CoV-2 provides an antigenic combination of the recognized viral proteins. In addition, with this inactivation method the viral proteins, such as the spike glycoprotein, seem to maintain their antigenicity when the conformation is preserved by the presence of the disulfide bonds.

As reported in the state of the art, the detected anti-SARS-CoV-2 antibodies IgG, IgM, and IgA appear as a consequence of the different stages of the COVID-19 disease. Therefore, their detection is important for the diagnosis, follow-up, and identification of asymptomatic cases. The immunoenzymatic tests of the invention were tested on a significant number of confirmed COVID-19 patients and negative samples, in parallel with reference assays, demonstrating the high sensitivity and specificity of the method for the detection of the 3 classes of antibodies IgG, IgM, and IgA.

### REFERENCES

[1] World Health Organization, Coronavirus disease 2019 (COVID-19), Situation Report—91.
[2] World Health Organization, COVID-19 Strategy Update, Apr. 14, 2020.
[3] World Health Organization, Laboratory testing for coronavirus disease (COVID-19) in suspected human cases: interim guidance, Mar. 19, 2020.
[4] Anna Petherick. Developing antibody tests for SARS-CoV-2. Lancet. 2020, 395(10230), 1101-1102.
[5] Sandeep Kumar Vashist. In Vitro Diagnostic Assays for COVID-19: Recent Advances and Emerging Trends. Diagnostics. 2020, 10(4), 202.
[6] Wanbing Liu, Lei Liu, Guomei Kou, Yaqiong Zheng, Yinjuan Ding, Wenxu Ni, Qiongshu Wang, Li Tan, Wanlei Wu, Shi Tang, Zhou Xiong, Shangen Zheng. Evaluation of Nucleocapsid and Spike Protein-based ELISAs for detecting antibodies against SARS-CoV-2. Journal of Clinical Microbiology March 2020, JCM.00461-20; DOI: 10.1128/JCM.00461-20.
[7] Andrea Padoan, Laura Sciacovelli, Daniela Basso, Davide Negrini, Silvia Zuin, Chiara Cosma, Diego Faggian, Paolo Matricardi, Mario Plebani. Clinica Chimica Acta 2020; DOI: 10.1016/j.cca.2020.04.026.
[8] Renhong Yan, Yuanyuan Zhang, Yaning Li, Lu Xia, Yingying Guo, Qiang Zhou. Structural basis for the recognition of SARS-CoV-2 by full-length human ACE2. Science 2020, 367, 1444-1448.
[9] Alexandra C. Walls, Young-Jun Park, M. Alejandra Tortorici, Abigail Wall, Andrew T. McGuire, David Veesler. Cell 2020, 180, 1-12.
[10] Nicole C. Ammerman, Magda Beier-Sexton, Abdu F. Azad. Growth and Maintenance of Vero Cell Lines. Curr Protoc Microbiol. 2008 November; APPENDIX: Appendix-4E.
[11] Valeria Cagno. SARS-CoV-2 cellular tropism. Lancet Microbe 2020 April, DOI: 10.1016/S2666-5247(20)30008-2.
[12] Reed, L. J.; Muench, H. A simple method of estimating fifty percent endpoints. The American Journal of Hygiene. 1938, 27, 493-497.
[13] Andrea Padoan, Francesco Bonfante, Matteo Pagliari, Alessio Bortolami, Davide Negrini, Silvia Zuin, Dania Bozzato, Chiara Cosma, Laura Sciacovelli, Mario Plebani. Analytical and clinical performances of five immunoassays for the detection of SARS-CoV-2 antibodies in comparison with neutralization activity. EBioMedicine 62 (2020) 103101
[14] Marzia Nuccetelli, Massimo Pieri, Francesca Gisone, Serena Sarubbi, Marco Ciotti, Massimo Andreoni, Sergio Bernardini. Evaluation of a new simultaneous anti-SARS-CoV-2 IgA, IgM and IgG screening automated assay based on native inactivated virus. International Immunopharmacology 92 (2021) 107330.

1. A method of detecting one or more anti-SARS-CoV-2 virus specific antibody in a biological sample, the method comprising:
a) incubating the biological sample with inactivated purified SARS-CoV-2 whole virus particles under suitable conditions to obtain a SARS-CoV-2 virus/antibody complex;
b) optionally washing to remove unbound material;
c) detecting said SARS-CoV-2 virus/antibody complex.

2. The method of claim 1, wherein the anti-SARS-CoV-2 virus antibody is an IgG and/or IgM and/or an IgA antibody.

3. The method of claim 1, wherein said inactivated purified SARS-CoV-2 whole virus particles are pre-immobilized to a solid support.

4. The method of claim 3, wherein the solid support is a chip, column matrix material, a culture plate, a tube, a dish, a flask, a microtiter plate, a bead, microsphere, reactor vessel, wells, a polystyrene paramagnetic particle (PMP) or a latex magnetic particle (LMP) or a combination thereof.

5. The method of claim 1, wherein the detecting step is performed by means of a labelled secondary antibody, preferably a monoclonal antibody.

6. The method according to claim 5, wherein the secondary antibody is labeled with a radioactive isotope, or an enzyme.

7. The method of claim 6, wherein the detecting step comprises measuring a signal from the label and comparing the signal to a control signal from a control biological sample known to be negative for SARS-CoV-2 virus antibodies.

8. The method according to claim 5 wherein said secondary antibody is an antibody against human antibodies or an antibody against a SARS-CoV-2 antigen.

9. The method according to claim 8 wherein said antibody against a SARS-CoV-2 antigen is an antibody directed to the Spike protein of SARS-CoV-2, e.g. S1, and/or said antibody has a neutralizing activity.

10. The method of claim 1, wherein said inactivated purified SARS-CoV-2 whole virus particles comprise at least one of the following proteins of SARS-CoV-2: virus spike glycoprotein, nucleocapsid protein, membrane protein, an envelope protein or an immunogenic fragment thereof.

11. The method of claim 1, wherein the biological sample is selected from the group consisting of blood, serum, plasma, body fluid, saliva and other secretions from the subject or tissue or cell extracts.

12. The method of claim 1, wherein the biological sample is obtained from the subject at least 3 days for IgM and IgA and at least 10 days for IgG after onset of symptoms of SARS-CoV-2 virus infection or after a risk of exposure to SARS-CoV-2 virus, or after the subject has recovered from SARS-CoV-2 virus infection.

13. The method of claim 1, wherein the subject has received a vaccine against SARS-CoV-2 virus and the biological sample is optionally obtained after administration of a vaccine dose.

14. The method of claim 1, further comprising a quantification of amount of specific SARS-CoV-2 virus antibodies in the biological sample.

15. The method of claim 14, wherein the amount of the SARS-CoV-2 virus antibody in the biological sample is proportional to the amount of the complex detected.

16. The method of claim 1, which is for the detection in a sample of antibodies against a variant of SARS-CoV-2 virus, such as English, South African or Brazilian variant.

17. A kit for working the method according to claim 1, comprising:
a) a solid support coated with inactivated purified SARS-CoV-2 whole virus particles,
b) at least one labeled secondary antibody able to bind to human IgG and/or IgM and/or IgA, or a labelled antibody anti-SARS-CoV-2 antigen;
c) a negative control;
d) a positive control;
e) a cut-off control or calibrator;
f) a sample diluent;
g) a substrate solution;
h) a washing solution; and
i) optionally a stop solution.

18. The method of claim 1, wherein said inactivated purified SARS-CoV-2 whole virus particles are particles of the SARS-CoV-2 strain 2019-nCoV/Italy-INMI1 (GenBank: SARS-CoV-2/INMI1-Isolate/2020/Italy: MT066156) or natural or recombinant derivative thereof.

19. (canceled)

20. The method of claim 4, wherein the solid support is a microtiter plate.

21. The method of claim 6, wherein the enzyme is peroxidase, alkalinephosphatase, β Galactosidase or acetylcholinesterase.
